(19) United States
(12) Patent Application Publication
(10) Pub. No.: US 2009/0048205 A1
(43) Pub. Date: Feb. 19, 2009

(54) COMBINATION THERAPY WITH SYNTHETIC TRITERPENOIDS AND GEMCITABINE

(76) Inventors: Colin Meyer, Frisco, TX (US); Michael Andreeff, Houston, TX (US); Marina Konopleva, Houston, TX (US); Robert M. Kral, JR., Grapevine, TX (US)

Correspondence Address: FULBRIGHT & JAWORSKI L.L.P., 600 CONGRESS AVE., SUITE 2400, AUSTIN, TX, 78701, US

(21) Appl. No.: 12/192,710

(22) Filed: Aug. 15, 2008

Related U.S. Application Data
(60) Provisional application No. 60/970,516, filed on Sep. 6, 2007.
(60) Provisional application No. 60/955,939, filed on Aug. 15, 2007.

(51) Int. Cl. A61K 31/7064; A61P 35/00

(52) U.S. Cl. 514/49

(57) ABSTRACT

The present invention concerns methods for treating cancer, such as pancreatic cancer, using combination therapies, including the combination of a synthetic triterpenoid, e.g., CDDO-Me, and gemcitabine.

The present application claims the benefit of priority to U.S. Provisional Application Nos. 60/970,516, filed Sep. 6, 2007, and 60/955,939, filed Aug. 15, 2007, the entire contents of each of which are incorporated by reference herein.

### BACKGROUND OF THE INVENTION

I. Field of the Invention

The present invention relates generally to the fields of biology and medicine. More particularly, it concerns compositions and methods for the treatment and prevention of cancer, including pancreatic cancer.

II. Description of Related Art

There are reported to be over 30,000 new diagnoses of pancreatic cancer in the United States every year, with a mortality approaching 99%. This gives pancreatic cancer the highest fatality rate of all cancers. Patients diagnosed with pancreatic cancer typically have a poor prognosis partly because the cancer usually causes no symptoms early on, leading to metastatic disease at the time of diagnosis. Fluorouracil, gemcitabine, and erlotinib are known chemotherapeutic drug agents used as palliative treatments for pancreatic cancer. Gemcitabine was approved by the U.S. Food and Drug Administration (FDA) in 1998 after a clinical trial reported improvements in quality of life in patients with advanced prostate cancer, marking the first FDA approval of a chemotherapy drug for a non-survival clinical trial endpoint.

Giving the mortality rate for pancreatic cancer and the limitations of the currently known chemotherapeutics for this and similar types of cancers, such as lung and ovarian, there exists a strong need for more effective treatments.

Separately, synthetic triterpenoids (TPs) have been developed as anti-inflammatory agents and their anti-inflammatory effects have been reported. Much of the research has focused on their chemotherapeutic potential. The connection between inflammation and carcinogenesis (Balkwill et al., 2005) led to synthesis and testing of anti-inflammatory triterpenoids for the treatment of cancer. The most potent of these agents, such as 2-cyano-3,12-dioxooleana-1,9(11)-dien-28-oic acid (CDDO), its methylester (CDDO-Me), and CDDO-Imidazolide (CDDO-Im), are some of the strongest known inhibitors of the de novo synthesis of inflammatory enzymes such as inducible nitric oxide synthase (iNOS) and inducible cyclooxygenase 2 (Honda et al., 1998; Honda et al., 1999; Suh et al., 1999; Honda et al., 2000; Bore et al., 2002; Honda et al., 2002; Place et al., 2003; U.S. Pat. Nos. 6,326,507, 6,552,075 and 6,974,801). The compounds are shown below.

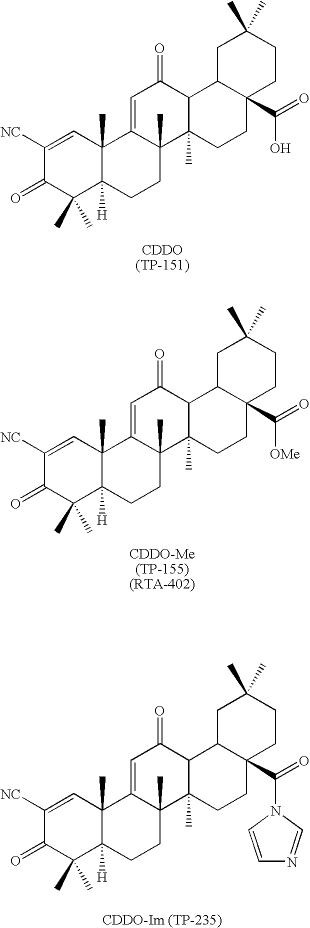

In addition to their anti-inflammatory actions, CDDO and its derivatives are also multifunctional compounds that induce differentiation, inhibit cell proliferation, and selectively induce apoptosis of a wide variety of cancer cells, including human lung cancer cells (Suh et al., 1999; Ito et al., 2000; Konopleva et al., 2002; Kim et al., 2002). Both CDDO and CDDO-Me are currently in phase I clinical trials for treatment of leukemia and solid tumors.

### SUMMARY OF THE INVENTION

The present invention overcomes limitation of the prior art by providing new combinations, methods and formulations for the treatment of cancer, including pancreatic cancer.

In one aspect, the invention provides a method for treating a cancer from a group consisting of pancreatic cancer, lung cancer and ovarian cancer, in a mammalian subject, comprising administering to said subject: a) a compound having the structure:

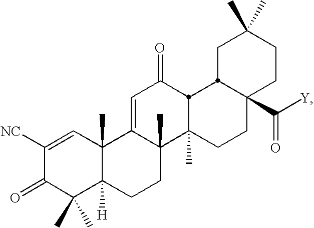

wherein Y is hydroxy, amino, or a heteroatom-substituted or heteroatom-unsubstituted C₁-C₃-alkoxy or C₁-C₃-alkylamino; or a pharmaceutically acceptable salt or hydrate thereof; and b) gemcitabine; wherein the combination is effective to treat the cancer.

Non-limiting examples of triterpenoids that may be used in accordance with the methods of this invention are shown here:

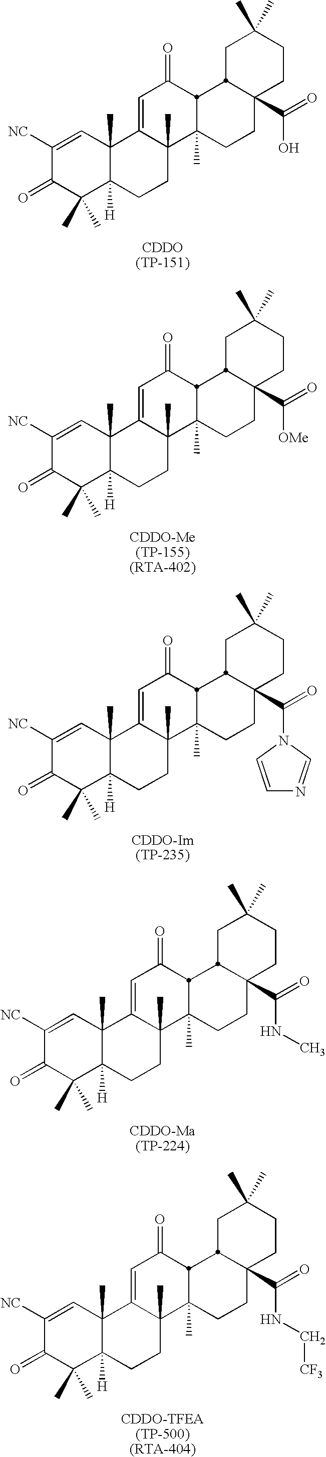

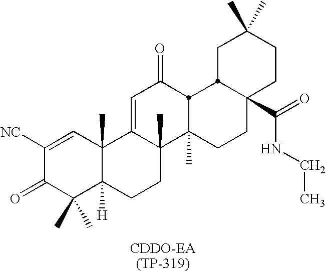

In some embodiments, the methods of the invention may be used to treat various stages of pancreatic cancer, including stage IV pancreatic cancer.

In some embodiments, the treatment results in an objective reduction of lesion size. In some variations of these embodiments, the objective reduction of lesion size is from about 10% to about 100%, from about 15% to about 50%, or from about 20% to about 35%. In certain embodiments, the objective reduction of lesion size is about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or higher, or any range derivable therein.

In some embodiments, the treatment results in the formation of no new metastases. In yet still further embodiments, treatment results in an increased white blood cell count in the subject relative to the white blood cell count of the subject in the absence of treatment. In some embodiments, the treatment results in an increased platelet count in the subject relative to the platelet count of the subject in the absence of treatment. Methods of measuring white blood cell counts and platelet counts are well known in the art.

In still further embodiments, Y, in the structure above, is a heteroatom-unsubstituted C₁-C₂-alkoxy group. In some of these embodiments, the compound is CDDO-methyl ester, for example, Form A of CDDO-methyl ester. In certain embodiments, the compound is provided in a daily dose from about 100 mg to about 600 mg, from about 150 to about 400 mg, or about 325 mg. In certain embodiments, the compound is provided in a daily dose of about, at least about, or at most about 50, 75, 100, 125, 150, 175, 200, 225, 250, 275, 300, 325, 350, 375, 400, 425, 450, 475, 500, 525, 550, 575, or 600 mg, or more, or any range derivable therein.

In other embodiments, the compound is an amorphous form of CDDO-methyl ester, for example, the compound may be a glassy solid form of CDDO-methyl ester, having an x-ray powder diffraction pattern with a halo peak at approximately 13.5° 2θ, as shown in FIG. 3C, and a T_{g}. The compound may be Form B of CDDO-Me. In certain aspects, the compound is provided in a daily dose from about 20 mg to about 200 mg, from about 30 mg to about 150 mg, or from about 30 mg to about 50 mg. In certain embodiments, the compound is provided in a daily dose from about, at most about, or at least about 10, 20, 30, 40, 50, 60, 70, 80, 90, 100, 110, 120, 130, 140, 150, 160, 170, 180, 190, 200 mg, or more, or any range derivable therein.

In another aspect of the invention, the amount of gemcitabine administered is the maximum tolerated dose (MTD). In other aspects, the amount of gemcitabine administered is from about 10% to about 90% of the maximum tolerated dose (MTD), from about 25% to about 75% of the MTD, or about 50% of the MTD. In particular embodiments, the amount of gemcitabine administered is from about 5%, 10%, 15%, 20%, 25%, 30%, 35%, 40%, 45%, 50%, 55%, 60%, 65%, 70%, 75%, 80%, 85%, 90%, 95%, 99%, or higher, or any range derivable therein, of the MTD.

In further aspects of the invention, the mammalian subject is a primate, such as a human. In other aspects. the mammalian subject is a cow, horse, dog, cat, pig, mouse, rat or guinea pig.

In another embodiment of the method, the CDDO-compound may be administered systemically. In other specific aspects of this embodiment, the CDDO-compound may be administered intravenously, intra-arterially, intra-peritoneally, orally, and/or during ex vivo bone marrow or blood stem cell purging. A CDDO compound, e.g., CDDO-Me, may be administered at daily dosages in the range of 0.1-30 mg/kg intravenously (i.v.) or 0.1-100 mg/kg orally, for example. In certain embodiments, about, at most about, or at least about 0.1, 0.2, 0.3, 0.4, 0.5, 1, 2, 3, 5, 10, 15, 20, 25, 30, 35, 40, 45, 50, 55, 60, 65, 70, 75, 80, 85, 90, 95, 100 mg/kg or higher, or any range derivable therein, of a CDDO compound may be administered by i.v. or may be administered orally. A CDDO compound, such as CDDO-Me, may be administered in the range of 0.1-100 mg/kg/day intravenously or 5-100 mg/kg/day orally for 3-30 days, for example. In certain embodiments, about, at most about, or at least about 0.1, 0.2, 0.3, 0.4, 0.5, 1, 2, 3, 5, 10, 15, 20, 25, 30, 35, 40, 45, 50, 55, 60, 65, 70, 75, 80, 85, 90, 95, or 100 mg/kg/day, or higher, or any range derivable therein, of a CDDO compound, such as CDDO-Me, may be administered by i.v. or about, at most about, or at least about 5, 10, 15, 20, 25, 30, 35, 40, 45, 50, 55, 60, 65, 70, 75, 80, 85, 90, 95, or about 100 mg/kg/day, or higher, or any range derivable therein, of a CDDO compound, such as CDDO-Me may be administered orally. The skilled artisan will appreciate that these dosages are only guidelines and a physician will determine exact dosages at the time of administration, factoring in other conditions such as age, sex, disease, etc., of the patient.

In another embodiment of methods of the present invention, gemcitabine, or a derivative thereof, may be administered systemically. In other specific aspects of this embodiment, gemcitabine, or a derivative thereof, may be administered intravenously, intra-arterially, intra-peritoneally, orally, and/or during ex vivo bone marrow or blood stem cell purging. Gemcitabine, or a derivative thereof, may be administered at daily dosages in the range of 0.1-30 mg/kg intravenously (i.v.) or 0.1-100 mg/kg orally, for example. In certain embodiments, about, at most about, or at least about 0.1, 0.2, 0.3, 0.4, 0.5, 1, 2, 3, 5, 10, 15, 20, 25, 30, 35, 40, 45, 50, 55, 60, 65, 70, 75, 80, 85, 90, 95, or 100 mg/kg (or higher, or any range derivable therein) of gemcitabine, or a derivative thereof, may be administered by i.v. or may be administered orally. Gemcitabine, or a derivative thereof, may be administered in the range of 0.1-100 mg/kg/day intravenously or 5-100 mg/kg/day orally for 3-30 days, for example. In certain embodiments, about, at most about, or at least about 0.1, 0.2, 0.3, 0.4, 0.5, 1, 2, 3, 5, 10, 15, 20, 25, 30, 35, 40, 45, 50, 55, 60, 65, 70, 75, 80, 85, 90, 95, or 100 mg/kg/day (or higher, or any range derivable therein) of gemcitabine, or a derivative thereof, may be administered by i.v. or about, at most about, or at least about 5, 10, 15, 20, 25, 30, 35, 40, 45, 50, 55, 60, 65, 70, 75, 80, 85, 90, 95, or 100 mg/kg/day (or higher, or any range derivable therein) of gemcitabine, or a derivative thereof, may be administered orally. The skilled artisan will appreciate that these dosages are only guidelines and a physician will determine exact dosages at the time of administration factoring in other conditions such as age, sex, disease, etc., of the patient.

The present invention also contemplates compositions and kits, such as compositions or kits comprising:

a) a compound having the structure:

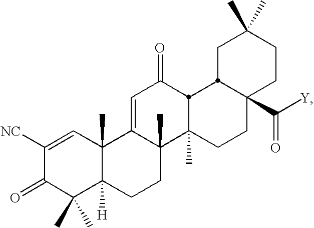

wherein Y is hydroxy, amino, or a heteroatom-substituted or heteroatom-unsubstituted C₁-C₃-alkoxy or C₁-C₃-alkylamino; or
a pharmaceutically acceptable salt or hydrate thereof; and

b) gemcitabine.

The composition may be a pharmaceutical composition, as discussed herein. In certain embodiments, Y is a heteroatom-unsubstituted C₁-C₂-alkoxy. A compound in the composition may be CDDO-Me, such as Form A of CDDO-Me or Form B of CDDO-Me. A compound within the composition may be an amorphous form of CDDO-Me. In certain embodiments, a compound within the composition is a glassy solid form of CDDO-Me, having an x-ray powder diffraction pattern with a halo peak at approximately 13.5° 2θ, as shown in FIG. 3C, and a T_{g}.

Any embodiment discussed herein with respect to one aspect of the invention applies to other aspects of the invention as well, unless specifically noted. For example, any composition of the invention may be used in any method of the invention, and any method of the invention may be used to produce or to utilize any composition of the invention. Any embodiment regarding a single compound as discussed herein is also contemplated as alternatively regarding a composition comprising two or more compounds, unless specifically noted otherwise.

Other objects, features and advantages of the present invention will become apparent from the following detailed description and any accompanying drawings. It should be understood, however, that the detailed description and any specific examples or drawings provided, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—White Blood Cell (WBC) Count Increases During CDDO-Me/Gemcitabine Combination Treatment. The white blood cell count is shown as function of treatment day (D) and treatment cycle (C) for two patients. Each treatment cycle consisted of 28 days, with 150 mg per day of CDDO-Me, given orally for 21 days, followed by seven days without drug. Then a new cycle followed. Also during each cycle, gemcitabine was administered once weekly, i.v., 1000 mg/m², three times per cycle (dosing on day 1, 8, and 15).

FIG. 2—Platelet Count (PLT) Increases During CDDO-Me/Gemcitabine Combination Treatment. The platelet count of two patients is shown as function of treatment day (D) and treatment cycle (C). Each treatment cycle consisted of 28 days, with 150 mg per day of CDDO-Me, given orally for 21 days, followed by seven days without drug. Then a new cycle followed. Also during each cycle, gemcitabine was administered once weekly, i.v., 1000 mg/m², three times per cycle (dosing on day 1, 8, and 15).

FIGS. 3A-C—X-Ray Powder Diffraction Pattern of Forms A and B of CDDO-Me. From top to bottom: unmicronized Form A (FIG. 3A); micronized Form A (FIG. 3B); and Form B (FIG. 3C).

### DESCRIPTION OF ILLUSTRATIVE EMBODIMENTS

### I. The Present Invention

The present invention concerns new methods and compounds for the treatment and prevention of diseases, including pancreatic cancer, involving the use of a novel combination therapy involving synthetic triterpenoids and gemcitabine.

The following patents and patent applications are incorporated herein by reference in their entirety: U.S. Ser. Nos. 09/998,009, 60/866,344, 60/916,273 and 60/955,939; U.S. Pat. Nos. 6,326,507, 6,552,075 and 6,974,801.

### II. Definitions

As used herein, the term “amino” means —NH₂; the term “nitro” means —NO₂; the term “halo” or “halide” designates —F, —Cl, —Br or —I; the term “mercapto” or “thio” means —SH; the term “cyano” means —CN; the term “azido” or “azo” means —N₃; the term “silyl” means —SiH₃, and the term “hydroxy” means —OH.

The term “alkyl” includes straight-chain alkyl, branched-chain alkyl, cycloalkyl (alicyclic), cyclic alkyl, heteroatom-unsubstituted alkyl, heteroatom-substituted alkyl, heteroatom-unsubstituted Cₙ-alkyl, and heteroatom-substituted Cₙ-alkyl. The term “heteroatom-unsubstituted Cₙ-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃(n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, and cyclohexyl, are all non-limiting examples of heteroatom-unsubstituted alkyl groups. The term “heteroatom-substituted Cₙ-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The following groups are all non-limiting examples of heteroatom-substituted alkyl groups: trifluoromethyl, —CH₂F, —CH₂Cl, —CH₂Br, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃.

The term “aryl” includes heteroatom-unsubstituted aryl, heteroatom-substituted aryl, heteroatom-unsubstituted Cₙ-aryl, heteroatom-substituted Cₙ-aryl, heteroaryl, heterocyclic aryl groups, carbocyclic aryl groups, biaryl groups, and single-valent radicals derived from polycyclic fused hydrocarbons (PAHs). The term “heteroatom-unsubstituted Cₙ-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C₆-C₁₀-aryl has 6 to 10 carbon atoms. Non-limiting examples of heteroatom-unsubstituted aryl groups include phenyl (Ph), methylphenyl, (dimethyl)phenyl, —C₆H₄CH₂CH₃, —C₆H₄CH₂CH₂CH₃, —C₆H₄CH(CH₃)₂, —C₆H₄CH(CH₂)₂, —C₆H₃ (CH₃)CH₂CH₃, —C₆H₄CH═CH₂, —C₆H₄CH═CHCH₃, —C₆H₄C≡CH, —C₆H₄C≡CCH₃, naphthyl, and the radical derived from biphenyl. The term “heteroatom-substituted Cₙ-aryl” refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C₁-C₁₀-heteroaryl has 1 to 10 carbon atoms. Non-limiting examples of heteroatom-substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, —C₆H₄CON(CH₃)₂, furanyl, thienyl, pyridyl, pyrrolyl, pyrimidyl, pyrazinyl, quinolyl, indolyl, and imidazoyl.

The term “alkoxy” includes straight-chain alkoxy, branched-chain alkoxy, cycloalkoxy, cyclic alkoxy, heteroatom-unsubstituted alkoxy, heteroatom-substituted alkoxy, heteroatom-unsubstituted Cₙ-alkoxy, and heteroatom-substituted Cₙ-alkoxy. The term “heteroatom-unsubstituted Cₙ-alkoxy” refers to a group, having the structure —OR, in which R is a heteroatom-unsubstituted Cₙ-alkyl, as that term is defined above. Heteroatom-unsubstituted alkoxy groups include: —OCH₃, —OCH₂CH₃, —OCH₂CH₂CH₃, —OCH(CH₃)₂, and —OCH(CH₂)₂. The term “heteroatom-substituted Cₙ-alkoxy” refers to a group, having the structure —OR, in which R is a heteroatom-substituted Cₙ-alkyl, as that term is defined above. For example, —OCH₂CF₃ is a heteroatom-substituted alkoxy group.

The term “alkylamino” includes straight-chain alkylamino, branched-chain alkylamino, cycloalkylamino, cyclic alkylamino, heteroatom-unsubstituted alkylamino, heteroatom-substituted alkylamino, heteroatom-unsubstituted Cₙ-alkylamino, and heteroatom-substituted Cₙ-alkylamino. The term “heteroatom-unsubstituted Cₙ-alkylamino” refers to a radical, having a single nitrogen atom as the point of attachment, further having one or two saturated carbon atoms attached to the nitrogen atom, further having a linear or branched, cyclic or acyclic structure, containing a total of n carbon atoms, all of which are nonaromatic, 4 or more hydrogen atoms, a total of 1 nitrogen atom, and no additional heteroatoms. For example, a heteroatom-unsubstituted C₁-C₁₀-alkylamino has 1 to 10 carbon atoms. The term “heteroatom-unsubstituted Cₙ-alkylamino” includes groups, having the structure —NHR, in which R is a heteroatom-unsubstituted Cₙ-alkyl, as that term is defined above. A heteroatom-unsubstituted alkylamino group would include —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —N(CH₃)₂, —N(CH₃)CH₂CH₃, —N(CH₂CH₃)₂, N-pyrrolidinyl, and N-piperidinyl. The term “heteroatom-substituted Cₙ-alkylamino” refers to a radical, having a single nitrogen atom as the point of attachment, further having one or two saturated carbon atoms attached to the nitrogen atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₁-C₁₀-alkylamino has 1 to 10 carbon atoms. The term “heteroatom-substituted Cₙ-alkylamino” includes groups, having the structure —NHR, in which R is a heteroatom-substituted Cₙ-alkyl, as that term is defined above.

As used herein, “water soluble” means that the compound dissolves in water at least to the extent of 0.010 mole/liter or is classified as water soluble according to literature precedence.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base such as methylamine, ethylamine, ethanolamine, lysine, ornithine and the like.

Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, Selection and Use (P. H. Stahl & C. G. Wermuth eds., Verlag Helvetica Chimica Acta, 2002), which is incorporated herein by reference.

Other abbreviations used herein are as follows: DMSO, dimethyl sulfoxide; iNOS, inducible nitric oxide synthase; COX-2, cyclooxygenase-2; NGF, nerve growth factor; IBMX, isobutylmethylxanthine; FBS, fetal bovine serum; GPDH, glycerol 3-phosphate dehydrogenase; RXR, retinoid X receptor; TGF-β, transforming growth factor-β; IFN-γ, interferon-γ; LPS, bacterial endotoxic lipopolysaccharide; TNF-α, tumor necrosis factor-α; IL-1β, interleukin-1β; GAPDH, glyceraldehyde-3-phosphate dehydrogenase; MTT, 3-[4,5-dimethylthiazol-2-yl]-2,5-diphenyltetrazolium bromide; TCA, trichloroacetic acid; HO-1, inducible heme oxygenase.

Compounds of the present invention may contain one or more asymmetric centers and thus can occur as racemates and racemic mixtures, single enantiomers, diastereomeric mixtures and individual diastereomers. In certain embodiments, a single diastereomer is present. All possible stereoisomers of the compounds of the present invention are contemplated as being within the scope of the present invention. However, in certain aspects, particular diastereomers are contemplated. The chiral centers of the compounds of the present invention can have the S- or the R-configuration, as defined by the IUPAC 1974 Recommendations. The present invention is meant to comprehend all such isomeric forms of the compounds of the invention. In certain embodiments, a compound is present in a mixture or a composition as predominantly one enantiomer.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s).

As used herein, “predominantly one enantiomer” means that the compound is present as at least 85% of one enantiomer, such as at least 90%, at least 95%, or at least 99% or more of one enantiomer. Similarly, compounds of the present invention may be “substantially free from other optical isomers,” meaning that the composition contains at most 5% of another enantiomer or diastereomer, such as at most 2% of another enantiomer or diastereomer, or at most 1% of another enantiomer or diastereomer.

Modifications or derivatives of the compounds disclosed throughout this specification are contemplated as being useful with the methods and compositions of the present invention. Derivatives may be prepared and the properties of such derivatives may be assayed for their desired properties by any method known to those of skill in the art.

In certain aspects, “derivative,” such as a gemcitabine derivative or a derivative of any of the compounds discussed herein, refers to a chemically modified compound that still retains the desired effects of the compound prior to the chemical modification. Such derivatives may have the addition, removal, or substitution of one or more chemical moieties on the parent molecule. Non-limiting examples of the types modifications that can be made to the compounds disclosed herein include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aryl group. Alternatively, in a cyclic or bicyclic structure (both aromatic and nonaromatic), heteroatoms such as N, S, or O can be substituted into the structure instead of a carbon atom to generate, for example, a heterocycloalkyl structure.

Prodrugs and solvates of compounds of the present invention are also contemplated herein. The term “prodrug” as used herein, is understood as being a compound which, upon administration to a subject, such as a mammal, undergoes chemical conversion by metabolic or chemical processes to yield a compound any of the formulas herein, or a salt and/or solvate thereof (Bundgaard, 1991; Bundgaard, 1985). Solvates of compounds of the present invention may be hydrates, for example.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the terms “patient” and “subject” are intended to include living organisms in which certain conditions as described herein can occur. Examples include humans, monkeys, cows, sheep, goats, dogs, cats, mice, rats, and transgenic species thereof. In a preferred embodiment, the patient is a primate. In certain embodiments, the primate or subject is a human. Other examples of subjects include experimental animals such as mice, rats, dogs, cats, goats, sheep, pigs, and cows. The experimental animal can be an animal model for a disorder, e.g., a transgenic mouse with a cancerous pathology. A patient can be a human suffering from cancer, such as pancreatic cancer.

“Treatment” and “treating” as used herein refer to administration or application of a therapeutic agent to a subject or performance of a procedure or modality on a subject for the purpose of obtaining a therapeutic benefit of a disease or health-related condition. For example, a subject (e.g., a mammal, such as a human) having cancer may be subjected to a treatment comprising administration of a compound or composition of the present invention.

The terms “inhibiting” or “reducing” or any variation of these terms as used herein includes any measurable decrease or complete inhibition to achieve a desired result. For example, there may be a decrease of 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of tumor size following administration of a compound or composition of the present invention.

The terms “contacted” and “exposed,” when applied to a cell, are used herein to describe the process by which an agent is delivered to a target cell or is placed in direct juxtaposition with a target cell. To achieve cell killing, for example, one or multiple agents are delivered to a cell in an amount or combined amount effective to kill the cell or prevent it from dividing. The terms “administered” and “delivered” are used interchangeably with “contacted” and “exposed.”

The term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When used in conjunction with the word “comprising” or other open language in the claims, the words “a” and “an” denote “one or more,” unless specifically noted. The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

### III. Synthetic Triterpenoids

Triterpenoids, biosynthesized in plants by the cyclization of squalene, are used for medicinal purposes in many Asian countries; and some, like ursolic and oleanolic acids, are known to be anti-inflammatory and anti-carcinogenic (Huang et al., 1994; Nishino et al., 1988). However, the biological activity of these naturally-occurring molecules is relatively weak, and therefore the synthesis of new analogs to enhance their potency was undertaken (Honda et al., 1997; Honda et al., 1998). Subsequent research has identified a number of synthetic compounds that have improved activity as compared to the naturally-occurring triterpenoids.

The ongoing efforts for the improvement of anti-inflammatory and antiproliferative activity of oleanolic and ursolic acid analogs led to the discovery of 2-cyano-3,12-dioxooleane-1,9(11)-dien-28-oic acid (CDDO) and related compounds (e.g., CDDO-Me, TP-225, CDDO-Im) (Honda et al., 1997, 1998, 1999, 2000a, 2000b, 2002; Suh et al., 1998; 1999; 2003; Place et al., 2003; Liby et al., 2005). In the case of inducing cytoprotective genes through Keap1-Nrf2-antioxidant response element (ARE) signaling, a recent structure activity evaluation of 15 triterpenoids confirmed the importance of Michael acceptor groups on both the A and C rings, the requirement for a nitrile group at C-2 of the A ring, and that substituents at C-17 dramatically affected pharmacodynamic action in vivo (Yates et al., 2007).

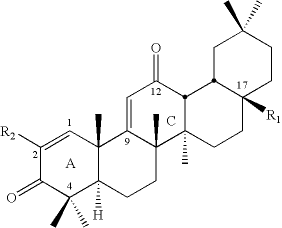

In general, CDDO is the prototype for a large number of compounds in a family of agents that have been shown useful in a variety of contexts. For example, CDDO-Me (CDDO methyl ester) and CDDO-Im are reported to possess the ability to modulate transforming growth factor-β (TGF-β)/Smad signaling in several types of cells (Suh et al., 2003; Minns et al., 2004; Mix et al., 2004). Both are known to be potent inducers of heme-oxygenase-1 and Nrf2/ARE signaling (Liby et al., 2005). For example, one important activity of the triterpenoids is their ability to activate the Keap/Nrf2/ARE pathway because activation of this phase 2 enzyme cytoprotective response is highly correlated to their anti-inflammatory activity (Liby et al., 2005, Dinkova-Kostova et al., 2005; Thimmulappa et al., 2006; Yu and Kensler, 2005; Na and Surh, 2006). In this regard, a series of synthetic triterpenoid (TP) analogs of oleanolic acid have also been shown to be potent inducers of the phase 2 response, that is, elevation of NAD(P)H-quinone oxidoreductase and heme oxygenase 1 (HO-1), which is a major protector of cells against oxidative and electrophile stress. See Dinkova-Kostova et al. (2005). Like previously identified phase 2 inducers, the TP analogs were shown to use the antioxidant response element-Nrf2-Keap1 signaling pathway.

Other pathways that CDDO-type compounds have been shown to affect include the blocking of NF-κB. It has been suggested that NF-κB activity may lead to enhancement of the cell cycle by its ability to activate cyclin D1 (Guttridge et al., 1999; Hinz et al., 1999; Joyce et al., 1999). Inhibition of IKK-driven NF-κB activation offers a strategy for treatment of different malignancies and can convert inflammation-induced tumor growth to inflammation-induced tumor regression. Luo et al., 2005, is incorporated herein by reference. For example, as reported by Shishodia et al. (2006), CDDO-Me modulates NF-κB activity and NF-κB-regulated gene expression. Using human leukemia cell lines and patient samples, it was shown that CDDO-Me potently inhibits both constitutive and inducible NF-κB activated by tumor necrosis factor (TNF), interleukin (IL)-1β, phorbol ester, okadaic acid, hydrogen peroxide, lipopolysaccharide, and cigarette smoke. NF-κB suppression occurred through inhibition of IκBα kinase activation, IκBα phosphorylation, IκBα degradation, p65 phosphorylation, p65 nuclear translocation, and NF-κB-mediated reporter gene transcription. This inhibition was shown to correlate with suppression of NF-κB-dependent genes involved in antiapoptosis (IAP2, cFLIP, TRAF1, survivin, and bcl-2), proliferation (cyclin d1 and c-myc), and angiogenesis (VEGF, cox-2, and mmp-9). CDDO-Me was also shown to potentiate the cytotoxic effects of TNF and chemotherapeutic agents. Overall, the results suggested that CDDO-Me inhibits NF-κB through inhibition of IκBα kinase, leading to the suppression of expression of NF-κB-regulated gene products and enhancement of apoptosis induced by TNF and chemotherapeutic agents.

In general, it is known that CDDO and its congeners form Michael adducts with thiol groups on cysteine residues of target proteins. Some of these such as Keap1 (Dinkova-Kostova et al., 2005), an inhibitor of the Nrf2 transcription factor that regulates the phase 2 cytoprotective response, and IκB kinase (Ahmad et al., 2006; Yore et al., 2006) have already been identified. Subsequent reports confirmed that CDDO-Me and CDDO-Im are direct inhibitors of IKKb activity, via binding to Cys179 (Ahmad et al., 2006; Yore et al., 2006). Given that triterpenoids form reversible Michael adducts with thiol groups, there are undoubtedly other targets, some of which may be implicated in the treatment effects presented in this application.

The aberrant or excessive expression of either inducible nitric oxide synthase (iNOS) or cyclooxygenase-2 (COX-2) has been implicated in the pathogenesis of many disease processes. For example, it is clear that NO is a potent mutagen (Tamir and Tannebaum, 1996), and that nitric oxide can also activate COX-2 (Salvemini et al., 1994). Furthermore, there is a marked increase in iNOS in rat colon tumors induced by the carcinogen, azoxymethane (Takahashi et al., 1997). A series of synthetic triterpenoid analogs of oleanolic acid have been shown to be powerful inhibitors of cellular inflammatory processes, such as the induction by IFN-γ of iNOS and of cyclooxygenase 2 in mouse macrophages. See Honda et al. (2000a); Honda et al. (2000b), and Honda et al. (2002), which are all incorporated herein by reference.

In animal models of many such conditions, stimulating expression of inducible heme oxygenase (HO-1) has been shown to have a significant therapeutic effect in many different diseases, including myocardial infarction, renal failure, transplant failure and rejection, stroke, cardiovascular disease, and autoimmune disease. See Sacerdoti et al., 2005; Abraham & Kappas, 2005; Bach, 2006; Araujo et al., 2003; Liu et al., 2006; Ishikawa et al., 2001; Kruger et al., 2006; Satoh et al., 2006; Zhou et al., 2005; Morse and Choi, 2005; and Morse and Choi, 2002. This enzyme breaks free heme down into iron, carbon monoxide (CO), and biliverdin (which is subsequently converted to the potent antioxidant molecule, bilirubin). It was shown that at nanomolar concentrations, CDDO and CDDO-Im rapidly increase the expression of the cytoprotective heme oxygenase-1 (HO-1) enzyme in vitro and in vivo. See Liby et al. (2005). Transfection studies using a series of reporter constructs showed that activation of the human HO-1 promoter by the triterpenoids requires an antioxidant response element (ARE), a cyclic AMP response element, and an E Box sequence. Inactivation of one of these response elements alone was shown to partially reduce HO-1 induction, but mutations in all three sequences entirely eliminated promoter activity in response to the triterpenoids.

Newer amide derivatives of CDDO have now also been found to be promising agents, for example for their ability to pass through the blood brain barrier, as discussed in greater detail below. In addition to the methyl amide of CDDO (CDDO-MA), as reported in (Honda et al., 2002), the invention provides additional CDDO amide derivatives, such as the ethyl amide (CDDO-EA), as well fluorinated amide derivative of CDDO, such as the 2,2,2-trifluoroethyl amide derivative of CDDO (CDDO-TFEA).

In general CDDO compounds can be prepared according to the methods taught by Honda et al. (1998), Honda et al. (2000b), Honda et al. (2002) and Yates et al. (2007), which are all incorporated herein by reference. For example, the synthesis of CDDO-MA is discussed in Honda et al. (2002). The syntheses of CDDO-EA and CDDO-TFEA are presented in Yates et al. (2007), which is incorporated herein by reference, and shown in the Scheme 1 below.

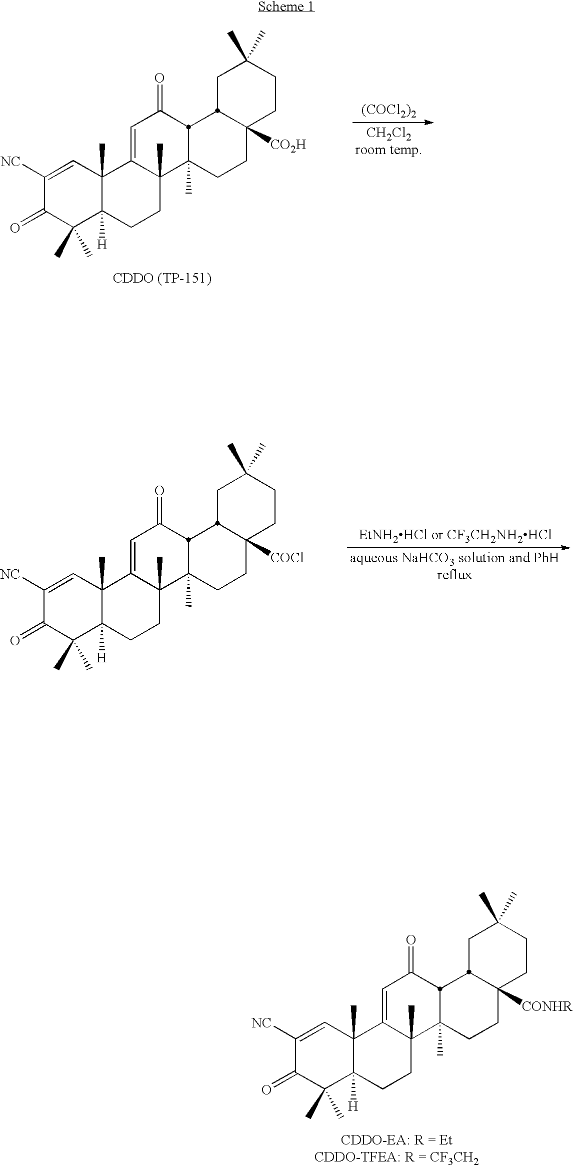

Given their structural similarity with other synthetic triterpenoids, such as CDDO-Me, these new CDDO derivatives, such as CDDO-TFEA and CDDO-EA are expected to have utility for the treatment and prevention of other diseases such as cancer (including pancreatic cancer), inflammation, Alzheimer's disease, Parkinson's disease, multiple sclerosis, autism, amyotrophic lateral sclerosis, rheumatoid arthritis, and inflammatory bowel disease, all other diseases whose pathogenesis is believed to involve excessive production of either nitric oxide or prostaglandins, and pathologies involving oxidative stress alone or oxidative stress exacerbated by inflammation.

For example, the invention contemplates that the treatment methods described herein may have one or more of the following properties: (1) the ability to induce apoptosis and differentiate both malignant and non-malignant cells, (2) activity at sub-micromolar or nanomolar levels as an inhibitor of proliferation of many malignant or premalignant cells, (3) the ability to suppress the de novo synthesis of the inflammatory enzyme inducible nitric oxide synthase (iNOS), (4) the ability to inhibit NF-κB activation, or (5) the ability to induce heme oxygenase-1 (HO-1).

### IV. Polymorphic Forms of CDDO-Me

“Form A” of CDDO methyl ester (CDDO-Me) is unsolvated (non-hydrous) and is characterized by a distinctive crystal structure, with a space group of P4₃ 2₁2 (no. 96) unit cell dimensions of a=14.2 Å, b=14.2 Å and c=81.6 Å, and by a packing structure, whereby three molecules are packed in helical fashion down the crystallographic b axis.

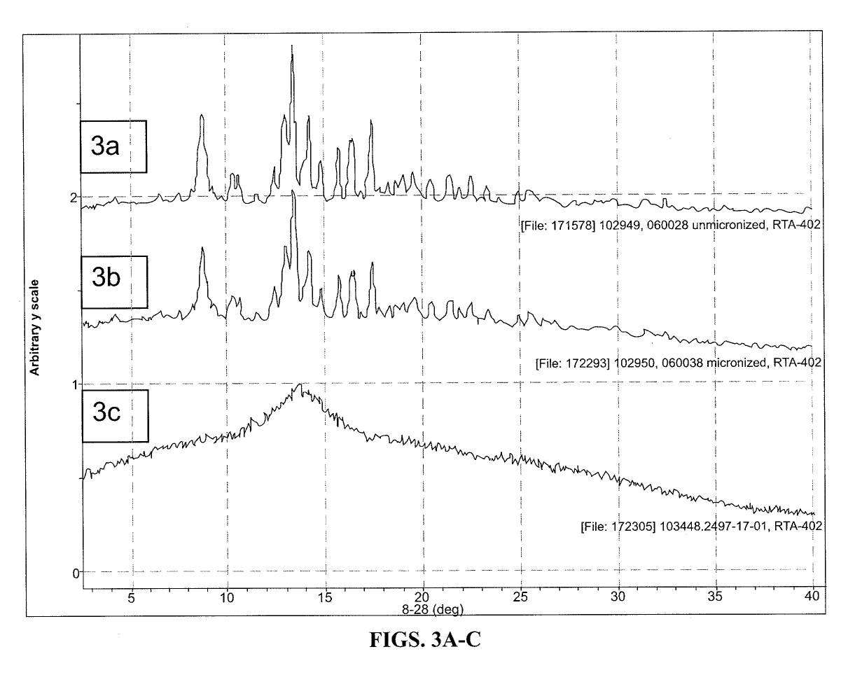

The other “Form B” of CDDO-Me is in a single phase but lacks such a defined crystal structure. Rather, Form B is typified by an x-ray powder diffraction (XRPD) spectrum differing from that of Form A (see FIG. 3). Moreover, Form B displays a bioavailability that is surprisingly better than that of Form A.

Methodology for the synthesis of CDDO methyl ester has been published. See U.S. Pat. No. 6,326,507, Honda et al. (1998), and Honda et al. (2000). Form A and Form B of CDDO methyl ester are readily prepared from a variety of solutions of the compound. In particular, Form B can be prepared by fast evaporation or slow evaporation in MTBE, THF, toluene, or ethyl acetate. By the same token, Form A can be prepared via fast evaporation, slow evaporation, or slow cooling of a CDDO methyl ester solution in ethanol or methanol. Preparations of CDDO methyl ester in acetone can produce either Form A, using fast evaporation, or Form B, using slow evaporation. Additional preparation methods are described below, including the tables provided there.

Since it does not have a defined crystal structure, Form B likewise lacks distinct XRPD peaks, such as those that typify Form A, and instead is characterized by a general “halo” XRPD pattern. In particular, the non-crystalline Form B falls into the category of “x-ray amorphous” solids because its XRPD pattern exhibits three or fewer primary diffraction halos. Within this category, Form B is a “glassy” material: As shown by the PDF, the nearest neighbor atom-atom interactions match that observed for crystalline Form A, but the notion of an average unit cell does not apply because there is no long-range order manifested.

Unlike Form A, therefore, samples of Form B show no long-range molecular correlation, i.e., above roughly 20 Å. Moreover, thermal analysis of Form B samples reveals a glass transition temperature (T_{g}). In contrast, a disordered nanocrystalline material, does not display a T_{g} but instead only a melting temperature (Tₘ), above which crystalline structure becomes a liquid.

The present description also characterizes a CDDO-methyl ester dimethanol solvate form that can be used to prepare Form B. Also characterized here is a CDDO-methyl ester hemibenzenate form.

Although micronization of other crystalline materials has been found to affect XRPD spectra, XRPD analysis of micronized Form A results in a spectrum similar to unmicronized Form A. See FIG. 3 for a side-by-side comparison of unmicronized Form A, micronized Form A, and Form B CDDO methyl ester.

Various means of characterization can be used together to distinguish Form A and Form B CDDO methyl ester from each other and from other forms of CDDO methyl ester. Illustrative of the techniques suitable for this purpose are solid state Nuclear Magnetic Resonance (NMR), X-ray powder diffraction, X-ray crystallography, Differential Scanning Calorimetry (DSC), dynamic vapor sorption/desorption (DVS), Karl Fischer analysis (KF), hot stage microscopy, modulated differential screening calorimetry, FT-IR, and Raman spectroscopy.

In particular, analysis of the XRPD and DSC data can distinguish Form A, Form B, and hemibenzenate forms of CDDO-methyl ester.

The properties of the inventive CDDO methyl ester forms are both distinctive, as mentioned above, and conducive to their use as medicinal agents. For example, the bioavailability of Form B and Form A CDDO methyl ester varied in monkeys when the monkeys received equivalent dosages of the two forms orally, in gelatin capsules. See Example 6. In addition, the stability of the newly identified CDDO-methyl ester forms will be useful in the production of pharmaceutical compositions.

The presence of multiple forms, including polymorphs, in pharmaceutical solids has been previously described, for instance, by Cui (2007). The crystalline and amorphous forms of a compound may exhibit different physical and chemical characteristics. For instance, amorphous forms may have higher solubility relative to the crystalline form. Every compound is unique in this regard, however, and the degree to which an amorphous material will differ from the crystalline state must be investigated on a case-by-case basis and cannot be predicted a priori. In addition, some amorphous materials are prone to re-crystallization.

In the present context, variability in data collection can arise due to a myriad of factors. Accordingly, this description uses the terms “about” or “approximately” to indicate variations in data used to describe the CDDO-methyl ester forms. For example, a melting temperature may vary based on instrumentation or conditions. Regarding the precision of the measurement, the U.S. Pharmacopeia Chapter 891 states that “In the case of melting, both an “onset” and a “peak” temperature can be determined objectively and reproducibly, often to within a few tenths of a degree.” Practical experience indicates this is not true for measuring the T_{g} of a material. The T_{g} will depend on many factors: how the sample was prepared, the thermal history of the sample (relaxation), residual solvent that may or may not volatilize prior to T_{g}, the instrument, sample preparation (sample mass, particle size, packing, diluents), the parameters used to measure T_{g} (particularly scan rate), the parameters used to determine the location of the T_{g} (onset temperature, mid-point temperature, inflection point temperature, or offset temperature), whether a relaxation endotherm is present at T_{g}, and other factors. Some factors will decrease T_{g} (plasticization due to residual water/solvent), while others will increase T_{g} (faster scan rate, relaxation) and may do so by as much as 10-15° C. The change in heat capacity at T_{g} (ΔCp) can be important, as reported by Zhou, 2002.

The present description speaks of different patterns in terms of their “characteristic” peaks. The assemblage or group of such peaks is unique to a given polymorphic form, within the uncertainty attributable to individual instruments and to experimental conditions, respectively.

For each of the crystalline forms, a group of five characteristic peaks is listed in Tables 17-19, below. Typical variation can be ±0.1° 2θ, but peak position can vary up to ±0.2° 2θ or more in some experiments.

The XRPD pattern of the glassy material (Form B) shows a broad halo peak at approximately 13.5° 2θ, which appears to be characteristic of Form B. Other halos are not as well-defined, and the shape/position of this pattern may change as a function of the instrument and experimental conditions. Variation in the position of this broad peak will be larger than that of the characteristic peaks of the respectively crystalline forms. In particular, variability of up to ±1° 2θ for the broad peak of Form B can be expected in certain instruments.

The present invention further relates to the use of Form A and Form B of CDDO methyl ester, respectively, for treating diseases associated with inflammation, including a cancerous condition and various pathologies affecting the central nervous system. Pursuant to the invention, treatment of these diseases comprises administering to a subject in need thereof an effective amount of the novel CDDO methyl ester forms enumerated here. These compounds have utility for ameliorating or preventing inflammation involved in the etiology of cancer, Alzheimer's disease (AD), Parkinson's disease (PD), multiple sclerosis (MS), amyotrophic lateral sclerosis (ALS), rheumatoid arthritis (RA) and other autoimmune diseases, inflammatory bowel disease, and other pathological conditions tied to excessive production of either nitric oxide or prostaglandins.

### V. Gemcitabine

A number of nucleoside analogs such as cytarabine, fludarabine, cladribine, capecitabine, gemcitabine and pentostatin are used clinically as highly effective anti-neoplastic agents. Among these, gemcitabine (2′,2′-difluoro-2′-deoxycytidine, Gemzar™) is of particular interest due to its unique activity against solid tumors and is presently used therapeutically to treat bladder, breast, lung, ovarian and pancreatic cancer. Gemcitabine is disclosed in U.S. Pat. Nos. 4,808,614 and 5,464,826, which are incorporated herein by reference for their teaching of how to synthesize, formulate, and use gemcitabine for treating susceptible neoplasms.

Several self-potentiating mechanisms unique to this nucleoside analog are believed responsible for the activity of gemcitabine against solid tumors. The diphosphate metabolite of gemcitabine inhibits ribonucleotide reductase, which results in lower concentrations of intracellular deoxycytidine triphosphate (dCTP) and thus, increased incorporation of the triphosphate gemcitabine metabolite into DNA, which results in inhibition of DNA synthesis and blocks completion of the cell division cycle. Additionally, reduction in dCTP concentration upregulates the enzyme cytidine kinase, which is responsible for initial phosphorylation of gemcitabine, a necessary step in the inhibition of DNA synthesis by the drug. Finally, the triphosphate metabolite of gemcitabine is an inhibitor of cytidine deaminase, which is responsible for gemcitabine inactivation by conversion to the uridine metabolite. Accordingly, the additive nature of the above factors may explain the efficacy of gemcitabine in treating solid tumors.

Synthetic derivatives of gemcitabine, including several prodrug compounds, have been previously described. See, for example, International Applications WO03/043631, WO01/21135, WO99/33483, WO98/32762, WO98/00173 and WO91/15498; U.S. Pat. Nos. 6,303,569, 5,606,048, 5,594,124, 5,521,294, 5,426,183 and 5,401,838; European Patents EP712860, EP0376518, EP577303, EP576230, EP329348 and EP272891; Alexander et al., 2003; Guo et al., 2001; Di Stefano et al., 1999; Guo et al., 1999; Chou et al., 1992; Richardson et al., 1992; and Baker et al., 1991. Any one or more of these derivatives may be utilized in the methods and compositions of the present invention.

Gemcitabine hydrochloride is typically administered by intravenous infusion at a dose of 1000 mg/m² over 20-45 minutes (for example, about 30 mg/m²/min) once weekly. Intravenous dosing schedules frequently follow 4-week cycles where the drug is administered weekly for 2, 3 or 4 consecutive weeks followed by a rest from treatment. The maximum tolerated dose of gemcitabine is 300 mg/kg/dose for mice and the maximum administered dose for humans is 1000 mg/m². Other salt forms can be utilized if desired, for example, the hydrobromide, monophosphate, sulfate, malonate, citrate, and succinate are readily prepared. Any dosing regimen described herein with respect to gemcitabine may be employed in methods of the present invention.

Suitable dosage ranges for oral administration are dependent on the potency of gemcitabine in the particular indication of interest as well as the prodrug bioavailability, but are generally about 100 mg-eq/m²/day to about 7000 mg-eq/m² of a compound. Dosage ranges may be readily determined by methods known to the artisan of ordinary skill.

Studies in preclinical animal models as well several clinical studies in patients with hematological and solid tumors have documented that prolonged, low-dose infusion of gemcitabine can show superior antitumor activity relative to bolus or shorter-term infusion schedules (for example, see Veerman et al., 1996; Tempero et al., 2003; Gandhi et al., 2002; Rizzieri et al., 2002; Patel et al., 2001; Maurel et al., 2001; Akrivakis et al., 1999). Doses of 10 mg/m²/min for up to 12 hours have been reported to be tolerated in patients. Long-term intravenous administration of gemcitabine itself is inconvenient for patients and requires close supervision by medical staff. Oral dosage of gemcitabine prodrugs of the type disclosed in U.S. Pat. No. 7,265,096, incorporated herein by reference, may offer advantages in providing gemcitabine exposure over prolonged time periods, while minimizing acute side effects associated with gastrointestinal toxicity of the drug.

### VI. Pharmaceutical Formulations and Routes of Administration

Compounds and compositions of the present invention may be administered by a variety of methods. In general, compounds and compositions of the present invention may be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, subcutaneously, subconjunctival, intravesicularlly, mucosally, intrapericardially, intraumbilically, intraocularally, orally, locally, systemically, via inhalation (e.g., aerosol inhalation), via injection, via infusion, via continuous infusion, via localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 1990). In particular embodiments, administration may be orally or by injection (e.g. subcutaneous, intravenous, intraperitoneal, etc.). In one embodiment, a compound or composition of the present invention may be administered locally. For example, the compound or composition may be administered by intratumoral injection and/or by injection into tumor vasculature.

Depending on the route of administration, one or more active compounds may be coated in a material to protect the compound from the action of acids and other natural conditions which may inactivate the compound. Such active compounds may also be administered by continuous perfusion/infusion of a disease or wound site, for example.

It is specifically contemplated that one compound of the present invention may be administered by one method, whereas a second compound is administered by a second method. Such methods of administration may be simultaneously or sequentially.

To administer the therapeutic compound by other than parenteral administration, it may be necessary to coat the compound with, or co-administer the compound with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., 1984).

The therapeutic compound may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. For example, pharmaceutical compositions of the present invention may comprise an effective amount of one or more compounds of the present invention or additional agents dissolved or dispersed in a pharmaceutically acceptable carrier. The phrases “pharmaceutically or pharmacologically acceptable” refers to molecular entities and compositions that do not produce an adverse, allergic or other untoward reaction when administered to an animal, such as, for example, a human, as appropriate. The preparation of a pharmaceutical composition that contains at least one candidate substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, a “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, pp 1289-1329, 1990). Except insofar as any conventional carrier is incompatible with the active ingredient, its use in the therapeutic or pharmaceutical compositions is contemplated. The candidate substance may comprise different types of carriers depending on whether it is to be administered in solid, liquid or aerosol form, and whether it need to be sterile for such routes of administration as injection.

The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It may be advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

Active compounds are administered at a therapeutically effective dosage sufficient to treat a condition in a patient. “Therapeutically effective amount” means that amount which, when administered to an animal for treating a disease, is sufficient to effect such treatment for the disease. A therapeutically effective amount may, for example, reduce the amount or severity of symptoms of a condition in a patient by at least about 20%, such as at least about 40%, 60%, or 80%, or more, relative to untreated subjects. For example, the efficacy of a compound can be evaluated in an animal model system that may be predictive of efficacy in treating the condition in humans, such as the model systems shown in the examples and drawings.

The actual dosage amount of a composition of the present invention administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

The dose can be repeated as needed as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art. For example, the time interval between doses may be about 1 hour to about 2 hours, about 2 hours to about 6 hours, about 6 hours to about 10 hours, about 10 hours to about 24 hours, about 1 day to about 2 days, about 1 week to about 2 weeks, or longer, or any time interval derivable within any of these recited ranges.

In certain embodiments, it may be desirable to provide a continuous supply of a pharmaceutical composition to the patient. This could be accomplished by catheterization, followed by continuous administration of the therapeutic agent. The administration could be intra-operative or post-operative.

In certain embodiments, pharmaceutical compositions may comprise, for example, at least about 0.1% of one or more compounds of the present invention. In other embodiments, one or more compounds of the present invention may comprise between about 2% to about 75% of the weight of the unit, or between about 25% to about 60%, for example, and any range derivable therein. In other non-limiting examples, a dose may also comprise from about 1, 5, 10, 50, 100, 200, 350, or about 500 microgram/kg/body weight, or about 1, 5, 10, 50, 100, 200, 350, 500, or 1000 mg/kg/body weight or more per administration, or any ranges derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

It is also contemplated that methods of the present invention may be employed after a subject has been previously treated with another anticancer agent, such as fluorouracil in the treatment of pancreatic cancer.

### VII. Evaluation of Treatment Methods

To monitor disease course and evaluate methods of treatment discussed herein, it is contemplated that the patients should be examined for appropriate tests every month. To assess the effectiveness of a drug or combination of drugs, a physician will determine parameters to be monitored depending on the type of cancer/tumor. Such parameters may involve methods to monitor reduction in tumor mass by, for example, computer tomography (CT) scans or magnetic resonance imaging (MRI) scans. Tests that may be used to monitor the progress of the patients and the effectiveness of the treatments include: physical exam, X-ray, blood work (e.g., testing for certain cancer markers), bone marrow work and other clinical laboratory methodologies.

Clinical responses may be defined by acceptable measure. For example, a complete response may be defined by complete disappearance of cancer cells, whereas a partial response may be defined by any value lower than 100% reduction of cancer cells, such as about, at least about, or at most about 95%, 90%, 80%, 70%, 60%, 50%, 40%, 30%, 20%, or 10%, or any range derivable therein. Also, as discussed herein, measures may involve assessing the objective reduction of lesion size in a subject. Other measurements may regard an increase in longevity of a subject, a reduction in pain experienced by the subject, a decrease in analgesic consumption by the patient, a lack of formation of any new metastases in a subject, an increase in white blood cell count in a subject, an increase in platelet count in a subject, a lack of recurrence of the cancer, or a delay in recurrence or metastasis of the cancer (e.g., a delay of about or at least about 2, 4, 6, 8, 10 months, or 1, 2, 3, 4, 5, 6, 7, 8, 9, 10, 11, 12, 13, 14, 15, 16, 17, 18, 19, 20, or more years, or any range derivable therein). Another measurement may be an analysis of Response Evaluation Criteria in Solid Tumors (RECIST) values over time, which are well-known criteria used to evaluate response to treatment in solid tumors. See Therasse et al. (2000), incorporated herein by reference. All of these measurement may be made in comparison to the condition of the patient in the absence of the treatment. Moreover, one or more of these measurements may be employed in methods of the present invention.

### VIII. Combination Therapy

Further elaboration of the combination therapy treatments provided and contemplated by this invention elaborated below. Such combination therapies may include the use of anti-inflammatory agents generally, or inhibitors of COX-2 and/or iNOS. Alternatively, the combination may be include a second or a third anti-cancer therapy, as discussed in detail below.

An “anti-cancer” agent is capable of negatively affecting cancer in a patient, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the synthetic triterpenoid (e.g., CDDO-Me) and the other agent(s) (e.g., gemcitabine) at the same time. This may be achieved by contacting the cell with a single composition or pharmacological formulation that includes both agents, or by contacting the cell with two distinct compositions or formulations, at the same time, wherein one composition includes the synthetic triterpenoid and the other includes the second agent(s), such as gemcitabine.

Alternatively, the synthetic triterpenoid therapy may precede or follow the other agent (e.g., gemcitabine) treatment by intervals ranging from minutes to weeks. In embodiments where the other agent and expression construct are applied separately to the cell, one would generally ensure that a significant period of time did not expire between the time of each delivery, such that the agent and the synthetic triterpenoid would still be able to exert an advantageously combined effect on the cell. In such instances, it is contemplated that one may contact the cell with both modalities within about 12-24 hours of each other and, more preferably, within about 6-12 hours of each other. In some situations, it may be desirable to extend the time period for treatment significantly, however, where several days (e.g., 2, 3, 4, 5, 6 or 7) to several weeks (e.g., 1, 2, 3, 4, 5, 6, 7 or 8) lapse between the respective administrations.

Various combinations may be employed, synthetic triterpenoid (e.g., CDDO-Me) therapy is “A” and the secondary agent, such as radio- or chemotherapy (e.g., gemcitabine), is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the synthetic triterpenoid compounds of the present invention to a patient will follow general protocols for the administration of chemotherapeutics, taking into account the toxicity, if any, of the drug. It is expected that the treatment cycles would be repeated as necessary. It also is contemplated that various standard therapies, as well as surgical intervention, may be applied in combination with the described hyperproliferative cell therapies. Non-limiting examples of such therapies are described below.

A. Chemotherapy

Cancer therapies may include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabine, navelbine, farnesyl-protein transferase inhibitors, transplatinum, 5-fluorouracil, vincristin, vinblastin and methotrexate, or any derivative of the foregoing.

B. Radiotherapy

Factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays may range from daily doses of 50 to 200 roentgens for prolonged periods of time (e.g., 3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

It has been shown that CDDO-Me can enhance the tumor-killing effect of radiation while simultaneously protecting normal tissue from radiation damage. This result is consistent with the anti-cancer effects and the protective effects against radiation-induced mucositis and chemotherapy-related toxicities other models shown in many animal models. These protective effects may be due to the Nrf2 activation and NF-κB inhibition. Therefore, the treatment methods of this invention, may be useful in enhancing the tumor-killing effect of radiation while simultaneously protecting normal tissue from radiation damage.

C. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with synthetic triterpenoid therapy. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erbB and p155.

D. Gene Therapy

In yet another embodiment, the secondary or tertiary treatment is a gene therapy in which a therapeutic polynucleotide is administered before, after, or at the same time as a synthetic triterpenoid. Therapeutic genes may include an antisense version of an inducer of cellular proliferation (sometimes called an oncogene), an inhibitor of cellular proliferation (sometimes called a tumor suppressor), or an inducer of programmed cell death (sometimes called a pro-apoptotic gene).

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. Methods of the present invention may therefore further comprise tumor resection in conjunction with administering one or more compounds of the present invention. The tumor resection may occur prior to the contacting of the tumor with a compound or composition of the present invention, for example. For example, the contacting can comprise treating a resected tumor bed with a triterpenoid and gemcitabine. In other aspects, tumor resection occurs after the contacting. In still other aspects, the contacting occurs both before and after tumor resection. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Anti-Inflammatory Agents

It is contemplated that other anti-inflammatory agents may be used in conjunction with the synthetic triterpenoid derivatives of the current invention. Other COX inhibitors may be used, including arylcarboxylic acids (salicylic acid, acetylsalicylic acid, diflunisal, choline magnesium trisalicylate, salicylate, benorylate, flufenamic acid, mefenamic acid, meclofenamic acid and triflumic acid), arylalkanoic acids (diclofenac, fenclofenac, alclofenac, fentiazac, ibuprofen, flurbiprofen, ketoprofen, naproxen, fenoprofen, fenbufen, suprofen, indoprofen, tiaprofenic acid, benoxaprofen, pirprofen, tolmetin, zomepirac, clopinac, indomethacin and sulindac) and enolic acids (phenylbutazone, oxyphenbutazone, azapropazone, feprazone, piroxicam, and isoxicam). (U.S. Pat. No. 6,025,395).

Histamine H2 receptor blocking agents may also be used in conjunction with the synthetic triterpenoid derivatives of the current invention, including cimetidine, ranitidine, famotidine and nizatidine.

### IX. Examples

The following examples are included to demonstrate specific embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Chemicals. Triterpenoids were synthesized as previously described in Honda et al. (2002), Honda et al. (1998), and Honda et al. (2000b). The various amide derivatives were synthesized by the condensation of CDDO acid chloride with the respective amine hydrochlorides (or free amines) using previously published methods Honda et al. (2002). The synthesis of CDDO-MA is discussed in Honda et al. (2002), which is incorporated herein by reference. The syntheses of CDDO-EA and CDDO-TFEA are presented in Yates et al. (2007), which is incorporated herein by reference, and shown in Scheme 1 above.

### Example 2

### Clinical Trial Results Using CDDO-Me and Gemcitabine

Dosage Information: RTA 402 dose: 150 or 300 mg per day (16% or 33% of maximum tolerated dose (MTD), respectively), given orally for 21 days, seven days without drug, then start a new cycle. Gemcitabine: administered once weekly, i.v., 1000 mg/m², three times per cycle (dosing on day 1, 8, and 15). This corresponds to a standard (MTD) regimen for gemcitabine. Patients were considered evaluable if they reached the end of cycle 2 without evidence of disease progression or severe adverse events. Radiological imaging was performed at the end of cycle 2 to assess drug activity.

Patients: All with Stage IV pancreatic cancer.

Results: Combination therapy was well tolerated, showing no signs of significant toxicity. A high percentage of evaluable patients (89%) experienced disease control (stable disease or objective response, the latter defined as at least a 30% reduction in overall target lesion burden, which entailed identifying lesion(s) for tracking over time and performing appropriate measurements of those lesions. See RECIST discussion in Therasse et al., (2000). Evidence of clinical activity was noted at both dose levels of RTA 402.67% of evaluable patients experienced measurable reductions in overall target lesion burden, and 33% experienced objective responses as evaluated using RECIST parameters. See Therasse et al., (2000). One patient who experienced a partial response received 14 cycles of therapy (150 mg per day, 21 days per 28 day cycle) before progressing. Because pancreatic cancer is typically quite difficult to treat, this level of drug effect is unusual (especially the percentage of objective responses). Historically, gemcitabine alone has not produced this level of clinical activity.

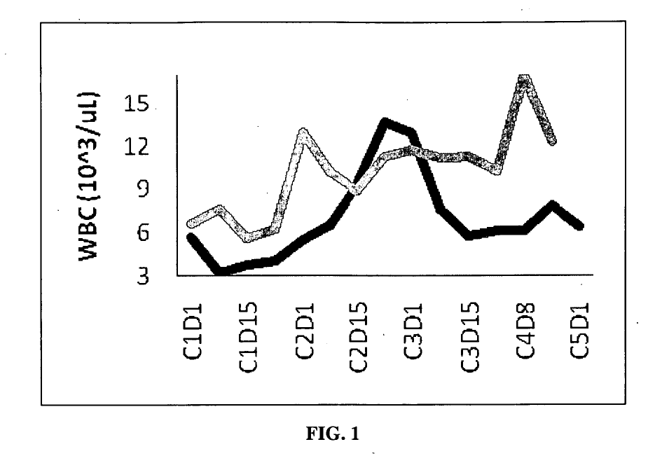

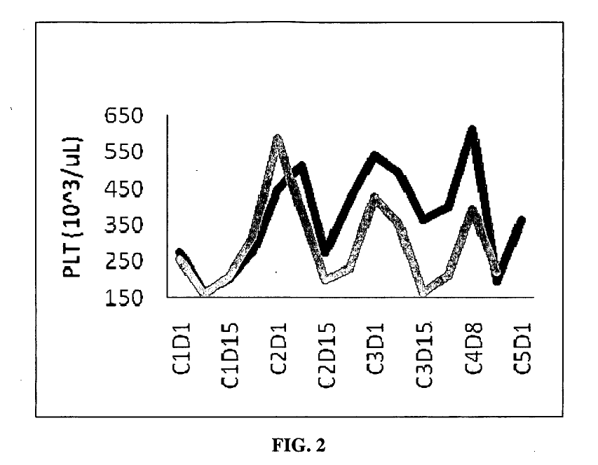

Blood work in a number of patients showed that the white blood cell counts and platelet counts went down significantly during the first week of cycle 1, had recovered by the end of week 3, and by the end of cycle 1 were approximately twice as high as the baseline counts. A similar pattern (initial reduction, recovery, and increase to or beyond the starting level) was noted in cycle 2. See FIG. 1 and FIG. 2.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,808,614
U.S. Pat. No. 5,401,838
U.S. Pat. No. 5,426,183
U.S. Pat. No. 5,464,826
U.S. Pat. No. 5,521,294
U.S. Pat. No. 5,594,124
U.S. Pat. No. 5,606,048
U.S. Pat. No. 6,025,395
U.S. Pat. No. 6,303,569
U.S. Pat. No. 6,326,507
U.S. Pat. No. 6,552,075
U.S. Pat. No. 6,974,801
U.S. Pat. No. 7,265,096
U.S. application Ser. No. 09/998,009
U.S. application Ser. No. 60/866,344
U.S. application Ser. No. 60/916,273
U.S. application Ser. No. 60/955,939
Abraham and Kappas, Free Radic Biol Med., 39(1):1-25, 2005.
Ahmad et al., J. Biol. Chem., 281:3576-3579, 2006.
Akrivakis et al., Anti-Cancer Drugs, 10:525-531, 1999.
Alexander et al., J. Med. Chem., 46:4205-4208, 2003.
Araujo et al., J Immunol., 171(3):1572-1580, 2003.
Bach, Hum Immunol. 67(6):430-432, 2006.
Bagasra et al., Proc. Natl. Acad. Sci. USA, 92:12041-12045, 1995.
Baker et al., J. Med. Chem., 34:1879-1884, 1991.
Balkwill et al., Cancer Cell, 7:211-217, 2005.
Beal, Curr. Opin. Neurobiol., 6:661-666, 1996.
Bore et al., Acta Cryst., C58:199-200, 2002.
Bundgaard, Drugs of the Future, 16:443-458, 1991.
Bundgaard, In: Design of Prodrugs, 7-9, 21-24, Elsevier, Amsterdam, 1985.
Chauhan and Chauhan, Pathophysiology, 13(3):171-181, 2006.
Chou et al., Synthesis, 565-570, 1992.
Coyle and Puttfarcken, Science, 262:689-695, 1993.
Cui et al., Intl. J. Pharmaceutics, 339:3-18, 2007.
Di Stefano et al., Biochem. Pharmacol., 57:793-799, 1999.
Dickerson et al., Prog. Neuropsychopharmacol Biol. Psychiatry., 31:551-556, 2007.
Dinkova-Kostova et al., Proc. Natl. Acad. Sci. USA, 102(12):4584-4589, 2005.
Dudhgaonkar et al., Eur J Pain. 2006 Aug. 18.
European Patent Appln. EP0376518
European Patent Appln. EP272891
European Patent Appln. EP329348
European Patent Appln. EP576230
European Patent Appln. EP577303
European Patent Appln. EP712860
Gandhi et al., J. Clin. Oncol., 20:665-673, 2002.
Genain and Nauser, J. Mol. Med., 75:187-197, 1997.
Gold et al., Brain, 129(8):1953-1971, 2006.
Guo et al., Cancer Chemother. Pharmacol., 48:169-176, 2001.
Guo et al., J. Org. Chem., 64:8319-8322, 1999.
Guttridge et al., Mol. Cell. Biol., 19:5785-5799, 1999.
Handbook of Pharmaceutical Salts: Properties, Selection and Use (Stahl and Wermuth, Eds.), Verlag Helvetica Chimica Acta, 2002.
Hanson et al., BMC Medical Genetics, 6(7), 2005.
Hinz et al., Mol. Cell. Biol., 19:2690-2698, 1999.
Honda et al., Bioorg. Med. Chem. Lett., 12:1027-1030, 2002.
Honda et al., Bioorg. Med. Chem. Lett., 19:2711-2714, 1998.
Honda et al., Bioorg. Med. Chem. Lett., 9:3429-3434, 1999.
Honda et al., J. Med. Chem., 43:1866-1877, 2000a.
Honda et al., J. Med. Chem., 43:4233-4246, 2000b.
Honda et al., Med. Chem. Lett., 7:1623-1628, 1997.
Honda et al., Org. Biomol. Chem., 1:4384-4391, 2003.
Huang et al., Cancer Res., 54:701-708, 1994.
Ishikawa et al., Circulation, 104(15):1831-1836, 2001.
Ito et al., Cell Growth Differ., 11:261-267, 2000.
Joyce et al., J. Biol. Chem., 274:25245-25249, 1999.
Juedes et al., J. Immunol., 164(1):419-426, 2000.
Kaltschmidt et al. Proc. Natl. Acad. Sci. USA, 94:2642-2647, 1997.
Kawakami et al., Brain Dev., 28(4):243-6, 2006.
Kendall-Tackett, International Breastfeeding J., 2(6), 2007.
Kim et al., Mol. Cancer. Ther., 1:177-184, 2002.
Konopleva et al., Blood, 99:326-335, 2002.
Kruger et al., J Pharmacol Exp Ther., Sep. 7, 2006.
Lee et al., Glia. 55(7):712-22, 2007.
Lencz et al., Molecular Psychiatry, 12(6):572-580, 2007.
Liby et al., Cancer Res., 65:4789-4798, 2005.
Liu et al., FASEB J., 20(2):207-216, 2006.
Luo et al., J. Clin. Invest., 115(10):2625-2631, 2005.
Maurel et al., Anti-Cancer Drugs, 12:713-717, 2001.
McGeer and McGeer, Brain Res. Brain Res. Rev., 21:195-218, 1995.
McGeer et al., Neurology, 19:331-338, 1996.
McIver et al., Pain., 120(1-2):161-9, 2005.
Merrill and Benvenist, Trends Neurosci., 19:331-338, 1996.
Minns et al., Gastroenterology, 127:119-26, 2004.
Mix et al., Mol. Pharmacol., 65:309-318, 2004.
Morris and Choi, J. Mol. Med., 80(2):96-104, 2002.
Morse and Choi, Am. J. Respir. Crit. Care Med., 172(6):660-670, 2005.
Na and Surh, Mol. Carcinog., 45:368-380, 2006.
Nishino et al., Cancer Res., 48:5210-5215, 1988.
Owens, Adv. Neurol., 98:77-89, 2006.
Patel et al., J. Clin. Oncol., 19:3483-3489, 2001.
PCT Appln. WO 01/21135
PCT Appln. WO 91/15498
PCT Appln. WO 98/00173
PCT Appln. WO 98/32762
PCT Appln. WO 99/33483
PCT Appln. WO 03/043631
Place et al., Clin. Cancer Res., 9:2798-2806, 2003.
Remington's Pharmaceutical Sciences, 18^{th} Ed., 1289-1329, 1990.
Richardson et al., Nucleic Acid Res., 20:1763-1769, 1992.
Rizzieri et al., J. Clin. Oncol., 20:674-679, 2002.
Ross et al., Nutr Neurosci., 6(5):277-81, 2003.
Ruster et al., Scand. J. Rheumatol., 34(6):460-3, 2005.
Sacerdoti et al., Curr. Neurovasc. Res., 2(2):103-111, 2005.
Salvemini et al., J. Clin. Invest., 93:1940-1947, 1994.
Sarchielli et al., Cephalalgia., 26(9):1071-9, 2006.
Satoh et al., Proc. Natl. Acad. Sci. USA, 103(3):768-773, 2006.
Shishodia et al., Clin. Cancer Res., 12(6):1828-1838, 2006.
Simonian and Coyle, Annu. Rev. Pharmacol. Toxicol., 36:83-106, 1996.
Stewart et al., Neurology, 48:626-632, 1997.
Strejan et al., J. Neuroimmunol., 7:27, 1984.
Suh et al., Cancer Res., 63:1371-1376, 2003.
Suh et al., Cancer Res., 58:717-723, 1998.
Suh et al., Cancer Res., 59(2):336-341, 1999.
Takahashi et al., Cancer Res., 57:1233-1237, 1997.
Tamir and Tannenbaum, Biochim. Biophys. Acta., 1288:F31-F36, 1996.
Tempero et al., J. Clin. Oncol., 21:3402-3408, 2003.
Therasse et al., J. Natl Cancer Instit., 92:205, 2000.
Thimmulappa et al., Biochem. Biophys. Res. Commun., 351:883-999, 2006.
Veerman et al., Cancer Chemother. Pharmacol., 38:335-342, 1996.
Virley, J. Amer. Soc. Exper. NeuroTherap., 2:638-649, 2005.
Vodovotz et al., In: Handbook of Experimental Immunology, Vols. I-IV, 1996.
Wermuth and Stahl, In: Pharmaceutical Salts: Properties, Selection and Use—A Handbook, Verlag Helvetica Chimica Acta, 2002.
Williams et al., Clin. Neurosci., 2(3-4):229-245, 1994.
Wolinsky et al., Ann. Neurol. 61(1):14-24, 2007.
Yates et al., Mol. Cancer. Ther., 6(1):154-162, 2007.
Yore et al., Mol. Cancer. Ther., 5:3232-3239, 2006.
Yu and Kensler, Mutat. Res., 591:93-102, 2005.
Zhou et al., Am J Pathol., 166(1):27-37, 2005.
Zhou, et al., J. Pharmaceutical Sciences, 91:1863, 2002.

What is claimed is:

1. A method for treating a cancer from a group consisting of pancreatic cancer, lung cancer and ovarian cancer, in a mammalian subject, comprising administering to said subject:
a) a compound having the structure: 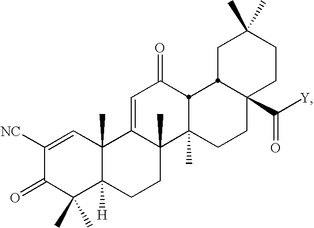
wherein Y is hydroxy, amino, or a heteroatom-substituted or heteroatom-unsubstituted C₁-C₃-alkoxy or C₁-C₃-alkylamino; or
a pharmaceutically acceptable salt or hydrate thereof; and
b) gemcitabine;
wherein the combination is effective to treat the cancer.

2. The method of claim 1, wherein the cancer is stage IV pancreatic cancer.

3. The method of claim 2, wherein the treatment results in an objective reduction of lesion size.

4. The method of claim 3, wherein the objective reduction of lesion size is from about 10% to about 100%.

5. The method of claim 4, wherein the objective reduction of lesion size is from about 15% to about 50%.

6. The method of claim 5, wherein the objective reduction of lesion size is from about 20% to about 35%.

7. The method of claim 2, wherein the treatment results in the formation of no new metastases.

8. The method of claim 2, wherein the treatment results in an increased white blood cell count in the subject.

9. The method of claim 2, wherein the treatment results in an increased platelet count in the subject.

10. The method of claim 1, wherein Y is a heteroatom-unsubstituted C₁-C₂-alkoxy.

11. The method of claim 10, wherein the compound is CDDO-Me.

12. The method of claim 11, wherein the compound is provided in a daily dose from about 100 mg to about 600 mg.

13. The method of claim 12, wherein the daily dose is from about 150 to about 400 mg.

14. The method of claim 13, wherein the daily dose is about 150 mg.

15. The method of claim 13, wherein the daily dose is about 300 mg.

16. The method of claim 11, wherein the compound is Form A of CDDO-Me.

17. The method of claim 11, wherein the compound is Form B of CDDO-Me.

18. The method of claim 11, wherein the compound is an amorphous form of CDDO-Me.

19. The method of claim 18, wherein the compound is a glassy solid form of CDDO-Me, having an x-ray powder diffraction pattern with a halo peak at approximately 13.5° 2θ, as shown in FIG. 3C, and a T_{g}.

20. The method of claim 1, wherein the amount of gemcitabine administered is the maximum tolerated dose (MTD).

21. The method of claim 1, wherein the amount of gemcitabine administered is from about 10% to about 90% of the maximum tolerated dose (MTD).

22. The method of claim 21, wherein the amount of gemcitabine administered is from about 25% to about 75% of the maximum tolerated dose (MTD).

23. The method of claim 23, wherein the amount of gemcitabine administered is about 50% of the maximum tolerated dose (MTD).

24. The method of claim 1, wherein the mammalian subject is a primate.

25. The method of claim 24, wherein the primate is a human.

26. The method of claim 1, wherein the mammalian subject is a cow, horse, dog, cat, pig, mouse, rat or guinea pig.
